(19) United States
(12) Patent Application Publication
(10) Pub. No.: US 2006/0195135 A1
(43) Pub. Date: Aug. 31, 2006

(54) PASS-THROUGH CATHETER

(76) Inventor: IHAB AYOUB, Houston, TX (US)

Correspondence Address: IHAB (AHAB) AYOUB, 2210 WEST DALLAS, APT 1632, HOUSTON, TX, 77019, US

(21) Appl. No.: 11/307,855

(22) Filed: Feb. 24, 2006

Related U.S. Application Data
(60) Provisional application No. 60/593,933, filed on Feb. 25, 2005.

(51) Int. Cl. A61M 29/00

(52) U.S. Cl. 606/192

(57) ABSTRACT

An apparatus is disclosed that includes an expandable member operatively connected to a catheter or similar device and includes at least one passage configured to fluidly connect the interior of a vessel or other body cavity regardless of the expansion state of the expandable member. Passages may be disposed in the expandable member, the catheter, the interface between the catheter and expandable member and any combination thereof. Passages may be configured such that flow therethrough is restricted or prevented when the expandable member is in an unexpanded state. A balloon having at least one of the described passage configurations is also disclosed. Methods for manufacturing and using such an apparatus are also disclosed.

### CROSS-REFERENCE TO RELATED APPLICATIONS

This application claims priority to U.S. Provisional Patent Application No. 60/593933, filed Feb. 25, 2005, entitled “PASS-THROUGH CATHETER” and hereby incorporated by reference in its entirety.

### BACKGROUND OF INVENTION

The invention relates generally to an apparatus for use in medical procedures.

### BACKGROUND ART

Catheters are traditionally inserted into body cavities or passages to allow fluids to pass into or out of the body, distend (expand) a target area, or convey diagnostic or other instruments. Certain types of catheters use a balloon or similar expandable component to achieve one of the aforementioned functions and/or to stabilize the catheter in a target area.

In use, such catheters will typically occlude part of a vessel or other passage. In angioplasty and stent placement procedures, such occlusion may be complete, such that flow through any such passage is prevented when the balloon is inflated. Depending on location and extent, such occlusion may have a number of negative effects on an organism. The effectiveness of such catheters is often limited due to the need for reduction in the extent or duration of occlusion during a procedure. Accordingly, a need exists for a device which may be effectively used in such procedures while reducing the extent of, and therefore the negative effects of, partial or total occlusion.

### SUMMARY OF INVENTION

In one embodiment, the invention comprises an apparatus comprising an expandable member operatively connected to a catheter or similar device and including at least one passage configured to be fluidly connected to the interior of a vessel in which the apparatus is disposed, the fluid connection occurring at both ends of the expandable member. Various embodiments include disposing the passage at least partially within the catheter, at least partially within the expandable member, and at least partially within both the expandable member and the catheter.

In one embodiment, the invention comprises an apparatus comprising an expandable member operatively connected to a catheter or similar device and including at least one passage fluidly connected to the environment at both ends of the expandable member, flow through which is enabled by at least a partial expansion of the expandable member.

In one embodiment, the invention comprises an apparatus comprising a plurality of expandable members operatively connected to a catheter or similar device and including at least one passage configured to be fluidly connected to the interior of a vessel in which the apparatus is disposed, the fluid connection occurring at one end of a first expandable member and extending to an opposite end of a second expandable member.

In one embodiment, the invention comprises a balloon for use with a catheter or similar device, the balloon comprising at least one passage in fluid communication with the environment at both ends of the balloon. The passage may be formed as a result of various configurations of the wall of the balloon and/or as a result of a separate longitudinal member operatively connected to the balloon.

In one embodiment, the invention comprises a method of manufacturing an apparatus including an expandable member and an operatively connected catheter or similar device, the method comprising forming at least one passage in the apparatus such that flow is enabled through that segment of the apparatus configured to at least partially occlude a portion of a biological organism in which the apparatus is disposed.

In one embodiment, the apparatus comprises a method of using an apparatus, including an expandable member, and operatively connected catheter or similar device, and a passage configured to permit flow past a predetermined portion of the apparatus when the apparatus is disposed in a biological organism and the expandable member at least partially expanded, the method comprising the positioning of at least one expandable member within a predetermined area of a biological organism and the expanding of the at least one expandable member such that flow through the area occupied by the expanded expandable member is enabled.

Other aspects and advantages of the invention will be apparent from the following description and the appended claims.

### BRIEF DESCRIPTION OF DRAWINGS

FIGS. 1A and 1B show an apparatus having a passage disposed largely within an interface according to one embodiment of the invention.

FIGS. 2A-2D are cross-sectional views of an apparatus having an expandable passage according to embodiments of the invention.

FIG. 3 shows flow through a passage of an apparatus according to one embodiment of the invention.

FIG. 4 shows an apparatus according to one embodiment of the invention.

FIGS. 5A-5C are cross-sectional views of various embodiments of the invention.

FIGS. 6A and 6B demonstrate the widening of an expandable passage according to one embodiment of the invention.

FIGS. 7A and 7B show an apparatus having a passage disposed largely within a catheter according to one embodiment of the invention.

FIGS. 8A-8C show an apparatus having a passage disposed at least partially within a catheter and partly within an expandable member according to embodiments of the invention.

FIGS. 9A and 9B show an apparatus having at least one passage that forms upon expansion of an expandable member according to one embodiment of the invention.

FIGS. 10A and 10B show an apparatus having multiple passages disposed longitudinally therethrough according to one embodiment of the invention.

FIGS. 11A and 11B show an apparatus having multiple passages disposed longitudinally therethrough according to one embodiment of the invention.

### DETAILED DESCRIPTION

In one embodiment, the invention comprises an expandable member and catheter combination configured such that vessel contents may pass through that length of vessel occupied by the expandable member when the expandable member is in an expanded state. In one embodiment, a passage is used to achieve and/or maintain flow through a predetermined area of vessel expected to be at least partially occluded by the expansion of an expandable member.

As used herein, an “expandable member” means any device or part thereof whose size, volume, and/or circumference may be expanded by an operator, or due to one or more signals from a second device. Expandable members include, but are not limited to, balloons of any configuration known in the art. The expansion may be reversible. Mechanisms of expansion include, but are not limited to, inflation using any substances known in the art, as well as any mechanical means. The definition of expandable member as used herein will also include any portion or segment of a catheter or similar device whose size, volume, and/or circumference may be expanded by an operator, or based on one or more signals from a second device. Other definitions of “expandable member” that are not incompatible with the spirit of the invention may also be applicable.

As used herein, “passage” is the configuration of any one or more elements described herein, either separately, or in combination, to permit flow through a region of vessel that is expected to be at least partially occluded by an expandable member when the expandable member is in an expanded state. Other definitions of “passage” that are not incompatible with the spirit of the invention may also be applicable.

As will be further described, a passage may be formed within a catheter (or similar device), an expandable member, or in the interface therebetween. A passage need not be continuously open to flow and it may be desirable that a passage is configured to be closed to flow when an expandable member is not in its expanded state, as flow will typically occur around an expandable member when not expanded.

As used herein, “proximal,” when used in reference to a side or end of an expandable member, means the side or end further from the leading end of a catheter to which the expandable member is operatively connected. As used herein, “distal,” when used in reference to a side or end of an expandable member, means the side or end closest to the leading end of a catheter to which the expandable member is operatively connected. The leading end of the catheter is that which first enters a predetermined location.

As used herein, a “catheter” means any longitudinal member for insertion into a body cavity, duct, or vessel. Other definitions of “catheter” that are not incompatible with the spirit of the invention may also apply.

As used herein, “interface” refers to the region between any part of a catheter (or similar device) and any part of an expandable member (e.g., a balloon) operatively connected thereto. Other definitions of “interface” that are not incompatible with the spirit of the invention may also apply.

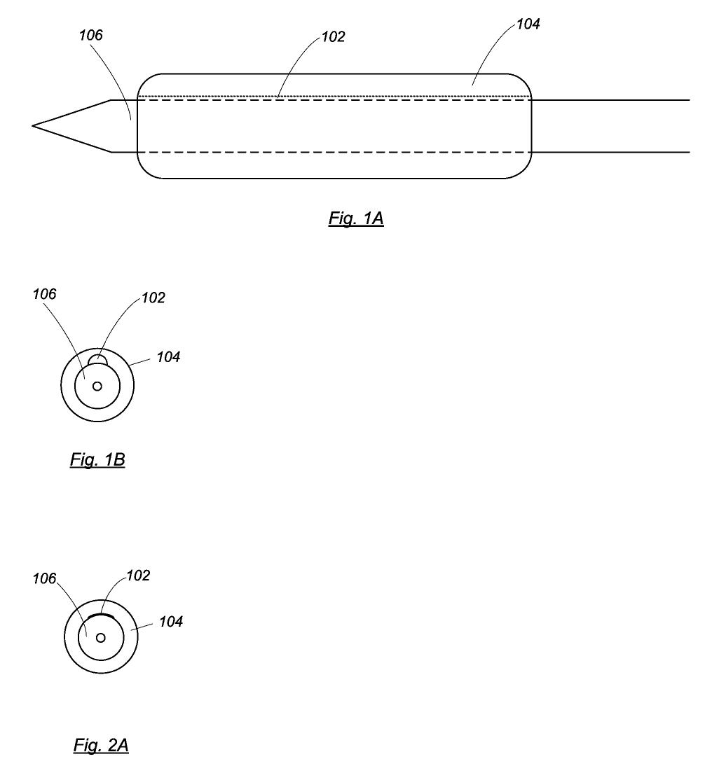

As shown in FIG. 1A, in one embodiment a passage 102, configured to be in fluid communication with the lumen of a vessel at either end of an expandable member 104, is formed in the interface between the expandable member 104 and a catheter 106 to which the expandable member is operatively connected. As shown in FIG. 1B, the passage 102 may be configured such that vessel contents may pass through it regardless of expansion state of the expandable member 104 (e.g., even in a non-expanded state). In one embodiment, such a configuration is achieved using a relatively rigid wall section of the expandable member 104 located at a predetermined interface between expandable member 104 and catheter 106. In one embodiment, such a configuration is achieved through the use of a separate longitudinal member disposed at the interface between expandable member 104 and catheter 106. Any other mechanism known in the art may also be used to create and/or maintain a longitudinal passage 102 along the interface between the expandable member 104 and catheter 106. Any number of passages 102 may be disposed in the interface between expandable member 104 and catheter 106.

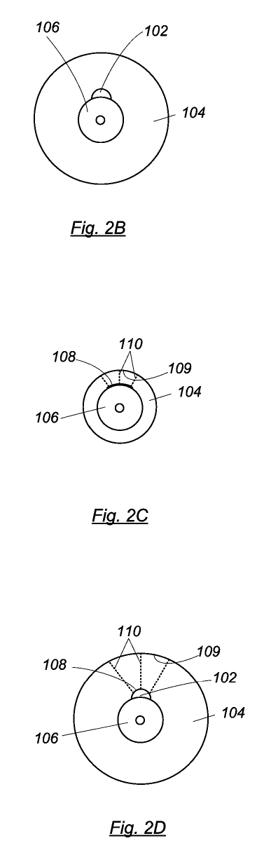

As shown in the embodiment of FIG. 2A, a passage 102 may also be configured such that the passage 102 is closed, or flow through the passage 102 restricted, when the expandable member 104 is in a non-expanded state. In such a configuration, the passage 102 may be formed as an area between the expandable member 104 and operatively connected catheter 106 where attachment of the expandable member 104 to catheter 106 does not occur, such that when the expandable member 104 is expanded, the section of expandable member 104 adjacent to the catheter 106 will be displaced away from the adjacent section of catheter 106 (as shown in FIG. 2B), thereby forming a longitudinal passage 102 between the expandable member 104 and catheter 106.

Formation of a passage 102 according to FIGS. 2A and 2B may occur in a number of ways. In one embodiment, the wall of the expandable member 104 in a predetermined area will be biased away from a surface of the catheter 106. In such a configuration, the non-expanded state will prevent the wall from taking the shape to which it is biased, while expansion of the expandable member 104 permits the wall to take the shape to which it is biased (allowing it to become displaced away from the adjacent section of catheter 106) resulting in formation of a passage 102. In one embodiment, vacuum pressure may be used to inhibit the tendency of the biased section of wall to take the shape toward which it is biased (e.g., vacuum pressure will keep the balloon folded tightly to the catheter 106 such that the passage walls are inhibited from moving in the direction toward which they are biased). Furthermore, as shown in the embodiment of FIGS. 2C and 2D, a first interior section 108 of the wall of the expandable member 104 may be connected by any means known in the art (connections represented at 109) to a second interior section 110 of the wall such that the two sections of wall 108, 110 are operatively connected, resulting in the displacement of the first section of wall 108 due to the outward movement of the second section of wall 110 caused by expansion of the expandable member 104. Any number of passages 102 may be formed in the interface between expandable member 104 and catheter 106.

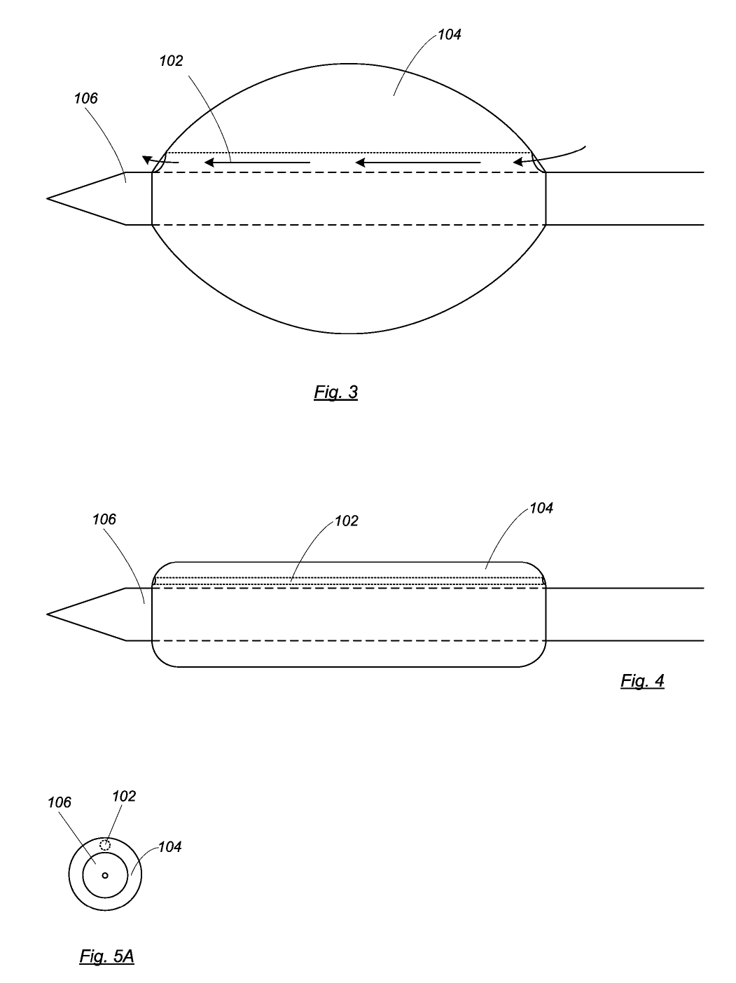

FIG. 3 shows one embodiment of an expanded expandable member 104 with arrows depicting flow through a passage 102 disposed at the interface between expandable member 104 and catheter 106. Although flow in this embodiment is depicted as occurring in a proximal to distal direction, flow may occur in any direction.

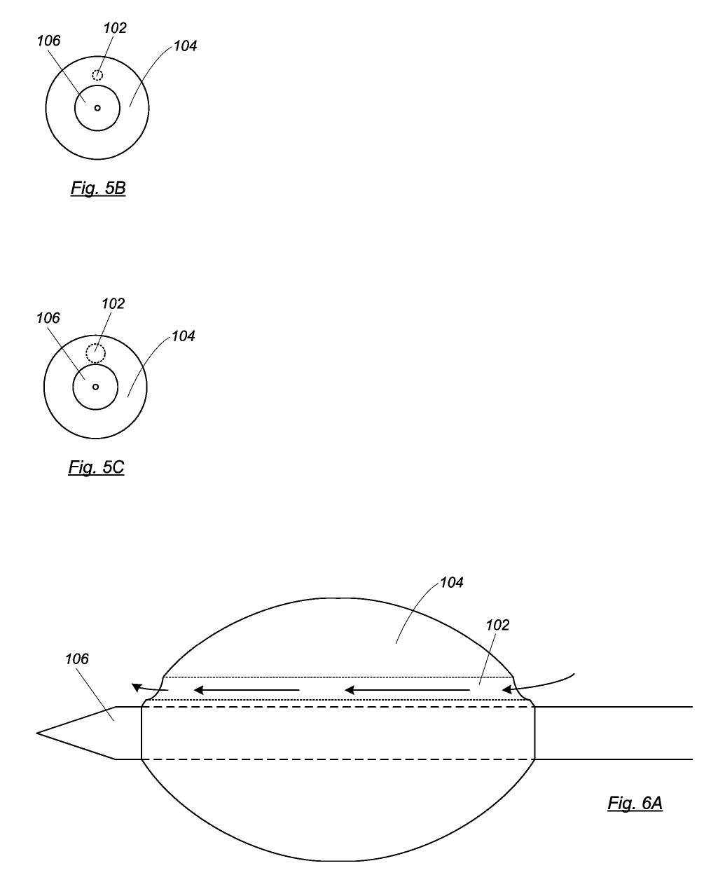

In the embodiment of FIG. 4, a passage 102 is formed within the expandable member 104 and may have a relatively fixed cross-section, as shown in FIGS. 5A-5B. In such an embodiment, the wall of the passage 102 may be formed of a relatively rigid material, and/or a separate elongate hollow member disposed at least partially within the expandable member 104. A relatively fixed cross-section advantageously permits flow through the passage 102 regardless of the expansion state of the expandable member 104.

In the embodiment of FIG. 5C, the width of the passage 102 increases upon expansion of the expandable member 104. In such an embodiment, the wall of the passage 102 may be constructed of a relatively elastic material that is compressed when the expandable member 104 is in a non-inflated state and expands into an open passage 102 as the expandable member 104 is inflated. In one embodiment, the wall of the passage 102 may be biased towards an expanded configuration, such expansion being hindered by a non-expanded state of the expandable member 104 in which the passage 102 is disposed. In one embodiment, the non-expanded state of the expandable member 104 may be maintained against the bias exerted by the wall of the passage 102 by use of vacuum pressure. Any number of passages 102 may be formed in the expandable member 104.

A passage 102 that is capable of expanding advantageously results in a reduced overall cross-section of the catheter/expandable member combination in the non-expanded state, while allowing increased flow through the passage 102 in the expanded state. A reduced cross-section of the catheter/expandable member combination advantageously facilitates the process of moving and/or positioning the expandable member 104 in a vessel.

An advantage of a cross-sectional area that increases during expansion of the expandable member 104 is the facilitation of increased flow through the passage 102 with increased expansion of the expandable member 104. Such proportional widening of the passage 102 will allow for increased flow in larger vessels because the expandable member 104 will typically also having an increased cross-section when fully expanded in larger vessels. Accordingly, those vessels that typically will have greater flow due to their size will not be overly restricted by the use of an expandable member having an expandable passage 102 as described herein.

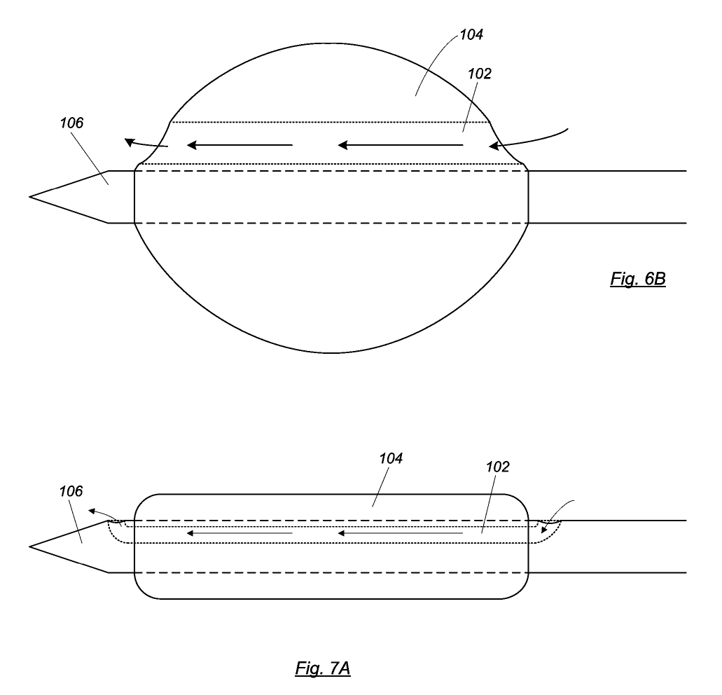

FIGS. 6A and 6B show how the cross-section of a passage 102 may increase in proportion to an increased cross-section (i.e., expansion) of an expandable member 104 according to one embodiment of the invention. As previously described, flow may occur in either direction (i.e., in either a proximal or distal direction), or alternatively may be limited to only one direction.

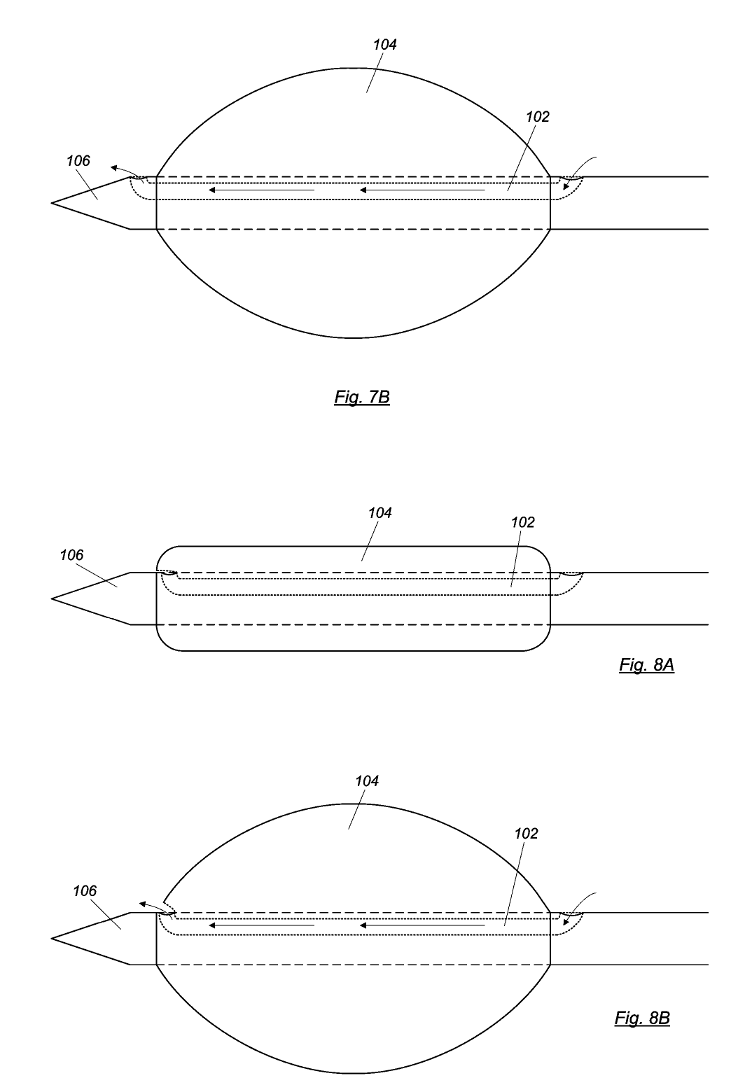

As shown in FIGS. 7A and 7B, in one embodiment a section of catheter 106 or similar device to which an expandable member 104 is operatively connected may include at least one passage 102 fluidly connected to the region surrounding the catheter 106 at both ends of the expandable member 104. The passage 102 may be formed as a tubular member at least partially disposed in the interior of the catheter 106. In one embodiment, the passage 102 is formed by configuring the interior of the catheter 106 such that openings disposed at either end of an operatively connected expandable member 104 are in fluid communication with each other within the catheter 106. Any other method known in the art may also be used to form a passage 102 through the catheter 106 that will enable flow through a section of vessel expected to be at least partially occluded due to the expansion of an expandable member 104. One embodiment may include a mechanism for sealing the passage 102 when flow therethrough is not desired. Any number of passages 102 may be disposed within the catheter 106 and flow may occur in either direction.

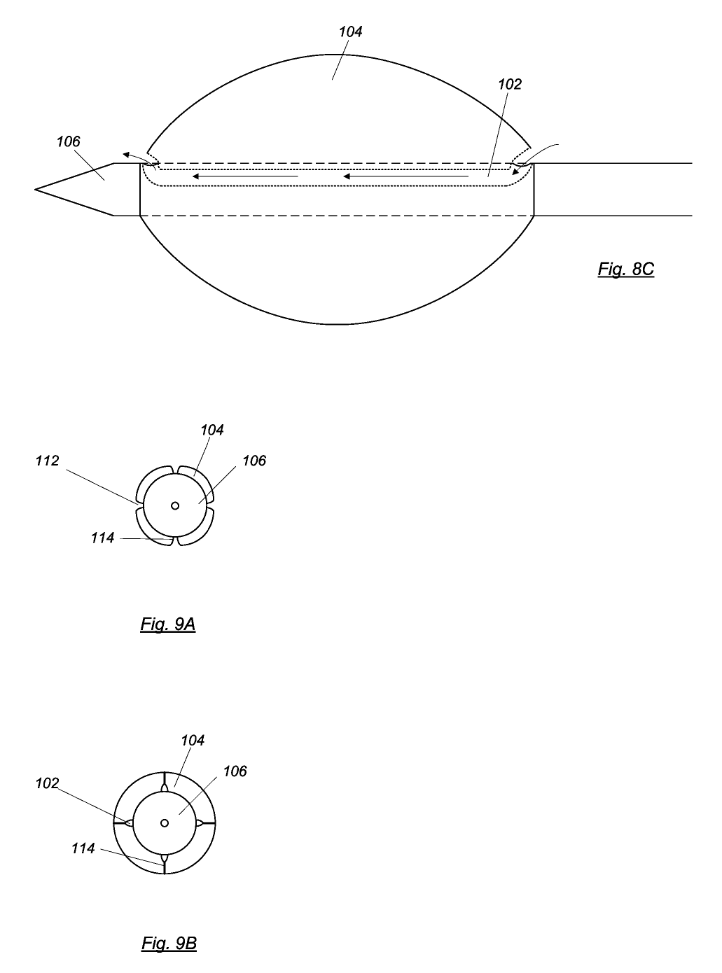

In the embodiment of FIGS. 8A and 8B, sections of a passage 102 are formed both in a catheter 106 and in the expandable member/catheter interface. In one embodiment, a passage 102 formed partially within the catheter 106 and partially in the interface of the expandable member 104 and catheter 106 may advantageously be sealed by a wall of the expandable member 104 when the expandable member 104 is not inflated (FIG. 8A) and open to flow when the expandable member 104 is at least partially inflated (FIG. 8B). In one embodiment, the passage 102 may be open regardless of inflation state of the expandable member 104. As in the other embodiments described herein, flow (depicted by arrows) may occur in either direction. Furthermore, the section of catheter 106 that is open to the vessel interior may be either distal or proximal relative to the expandable member 104 with the section that opens into the expandable member/catheter interface disposed at the other side of the expandable member 104. As shown in the embodiment of FIG. 8C, all openings into a passage 102 disposed largely within the catheter 106 may open into the catheter/expandable member interface such that when the expandable member 104 is in an unexpanded state, all openings into the passage 102 are sealed by the wall of the expandable member 104.

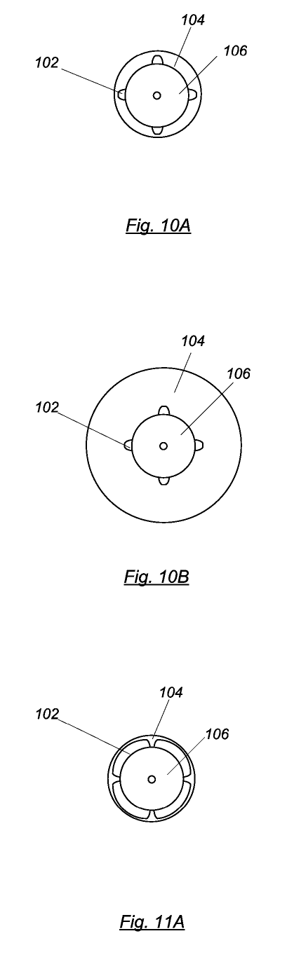

In the embodiment shown in FIGS. 9A and 9B, a plurality of expandable members 104, and/or at least one multi-lobed expandable member 104, may be configured such that gaps 112 between the expandable members 104 or lobes of a multi-lobed expandable member 104 will occur between relatively rigid or biased side walls 114. Upon expansion of the expandable member 104, as shown in FIG. 9B, the gaps 112 will form passages 102 between adjacent expandable members 104 or lobes thereof. The use of relatively rigid or biased wall sections 114 maintains the gaps that form passages 102 in the expanded state and prevents adjacent expandable members 104 or lobes thereof from occluding the space occupied by the gaps 112.

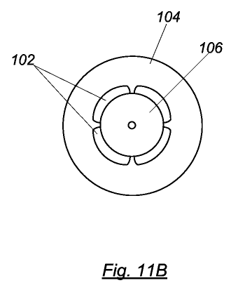

FIGS. 10A and 10B show examples of an embodiment having multiple passages 102 that extend longitudinally along the length of the expandable member 104. FIGS. 11A and 11B show examples of an embodiment in which passages 102 are disposed along a majority of the circumference of the section of catheter 106 adjacent to an operatively connected expandable member 104. Such a configuration advantageously provides for a greater volume of flow through the passages 102 due to their increased width. The passages 102 according to the embodiments of FIGS. 10A-B and 11 A-B may have a fixed or expandable cross-section. Furthermore, the passages 102 according to these embodiments may be formed by any of the approaches described herein.

In one embodiment, the invention comprises a balloon configured to form a passage through a predetermined section of a vessel. In one embodiment, the invention comprises a balloon having a wall section configured to form and/or accommodate a passage in an interface formed by operatively connecting the balloon to a catheter or similar apparatus. In one embodiment, the invention comprises a balloon having a longitudinal member disposed at least partially therein such that a passage is formed through the balloon substantially parallel to the longitudinal axis of the catheter. In one embodiment, the invention comprises a balloon having multiple passages formed therein according to one or more of the configurations described herein.

In one embodiment, multiple passages 102 are used according to any of the configurations described herein. Embodiments of the invention may also be used with multiple expandable members 104. In one embodiment, a passage 102 may be configured to provide flow past a plurality of expandable members 104 operatively connected to the catheter 106.

In one embodiment, flow may occur past multiple expandable members 104 by the disposition of at least one passage 102 through a portion of a catheter 106 to which the multiple expandable members 104 are operatively connected. In one embodiment, a passage 102 configured to allow flow past one expandable member 104 may be fluidly connected to a second passage 102 configured to allow flow past a second expandable member 104. One or more of these embodiments would advantageously provide a mechanism for the flow of fluids through an area of vessel in which multiple expandable members 104 are being used, while allowing for the creation of a restricted space between those expandable members 104 for the application of drugs or sampling of biological contents within the space between any two inflated expandable members 104.

In one embodiment, a passage 102 may include a filtering mechanism configured to filter predetermined substances flowing through the passage 102. In one embodiment, a mechanism may be used in conjunction with a filter to shunt the predetermined filtrates to a predetermined location. In one embodiment, the passage 102 may comprise a substantial portion of the catheter 106 lumen. For example, the interior of the catheter 106 may be configured such that opening as either end of an operatively attached balloon 104 are in fluid communication therethrough. A separate passage for a guide wire may be provided through such an area, such that the guide wire is isolated from vessel contents passing through the interior of the catheter 106.

Expandable members 104 and/or catheters 106 according to various embodiments of the invention may comprise any material known in the art. Embodiments of the invention need not be limited to use with a typical catheter 106 but may also be used with other devices used to position and/or stabilize an expandable member 104 (or other obstructing device) in a vessel. The various configurations of passages 102 described herein may be used in any combination. Furthermore, each of the various elements described herein may be of any size, and comprise any one or more materials known in the art.

Various components and compounds may be used to facilitate the successful operation of a passage 102 according to one or more embodiments of the invention. For instance, special coatings or compounds may be used to lessen the occurrence of blockage of the passage 102. Such coatings or compounds may include, but are not limited to, those having an anticoagulant effect on the contents of the vessel and those that may otherwise facilitate flow through the passage 102.

Embodiments of the invention may be used with any expandable member catheter configuration, including but not limited to those used to deliver drugs, sample vessel contents, interact with plaque or other vessel contents, insert a stent, and/or any other use known in the art. Furthermore, passages 102 may be configured such that flow therethrough may occur in either direction (i.e., distal to proximal and proximal to distal) and may include any mechanism known in the art (e.g., one-way valves) to permit flow in only one direction.

In one embodiment, the invention comprises a method of manufacturing a combination catheter and expandable member. A catheter is provided and an expandable member is either integrated into or otherwise operatively connected to the catheter. In one embodiment at least one passage is formed at the interface between the catheter and an operatively connected expandable member. In one embodiment, the expandable member comprises at least one passage that will be in fluid communication with the vessel contents on both the proximal and distal sides of the expandable member when the expandable member is disposed in the vessel and at least partially expanded. In one embodiment, the catheter includes at least one passage that will be in fluid communication with the vessel contents on both the proximal and distal sides of the expandable member when the expandable member is disposed in the vessel and at least partially expanded. In one embodiment, at least one passage at least partially disposed in the expandable member will be in fluid communication with at least one passage at least partially disposed within the catheter such that vessel contents may flow from one end of the expandable member to a second end of the expandable member when the expandable member is at least partially expanded. In one embodiment, the passage is configured such that flow through the passage is restricted when the expandable member is not at least partially expanded.

In one embodiment, the invention comprises a method of using a combination catheter and expandable member. At least one passage is disposed in the combination catheter and expandable member such that fluid communication through a section of vessel that will be at least partially occluded by expansion of the expandable member may occur through the passage. In one embodiment, an operator will position at least one expandable member in a predetermined section of a vessel of a biological organism using a catheter or similar apparatus to guide and/or position the expandable member. Once the expandable member is properly positioned in the predetermined section of vessel, the operator will initiate expansion of the expandable member and flow of vascular contents through a passage disposed in the catheter/expandable member combination will occur.

Configurations depicted in the drawings are intended to be merely representative of various configurations, and are not necessarily representative of any particular style or configuration of a balloon and/or catheter. Configurations and/or dimensions of balloons and/or catheters may be of any type known in the art. Furthermore, the drawings are not necessarily to scale, relative or absolute (e.g., catheter diameter relative to balloon diameter, passage diameter relative to catheter and/or balloon diameter, balloon length, balloon expandability, etc.).

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

What is claimed is:

1. An apparatus for flow through a portion of a lumen, comprising:
a catheter;
at least one expandable member operatively connected to the catheter; and
at least one passage configured to be fluidly connected to the lumen on both a distal and a proximal side of the expandable member.

2. The apparatus of claim 1, wherein flow through the at least one passage is enabled by at least a partial expansion of the at least one expandable member.

3. The apparatus of claim 1, wherein the at least one passage is at least partially disposed in the catheter.

4. The apparatus of claim 1, wherein the at least one passage is disposed in the at least one expandable member.

5. The apparatus of claim 1, wherein the at least one passage is disposed within an interface between the at least one expandable member and the catheter.

6. The apparatus of claim 1, wherein the at least one passage is disposed partly within the catheter and partly within an interface between the at least one expandable member and the catheter.

7. A balloon for use with a catheter, comprising:
a section configured to form a passage.

8. The balloon of claim 7, wherein the passage is disposed such it will lie between the balloon and the catheter when the balloon and the catheter are operatively connected.

9. The balloon of claim 7, wherein the section comprises a relatively inflexible material such that the passage will be open to flow regardless of inflation state of the balloon.

10. The balloon of claim 7, wherein the section comprises a material configured to create a passage only when the balloon is at least partially inflated.

11 . The balloon of claim 7, wherein the passage is disposed such that it will be fluidly connected to a second passage disposed in the catheter adjacent to the balloon.

12. The balloon of claim 11, wherein the fluid connection is open to flow only when the balloon is at least partially inflated.

13. The balloon of claim 11, wherein a separate elongate member is disposed in the passage.

14. The balloon of claim 7, wherein the balloon comprises a plurality of lobes.

15. The balloon of claim 7, wherein the section configured to form a passage is disposed between a plurality of connection points configured to operatively connect the balloon to the catheter.

16. A method of manufacturing an apparatus including a catheter and expandable member, comprising:
providing an expandable member;
providing a catheter; and
disposing a passage such that it will allow flow through at least one selected from the expandable member, the catheter, and an interface substantially disposed between the expandable member and the catheter.

17. The method of claim 16, wherein the disposing is concurrent with operatively connecting the expandable member to the catheter.

18. The method of claim 16, wherein the disposing occurs during the manufacture of the expandable member.
